(19) United States
(12) Patent Application Publication
(10) Pub. No.: US 2022/0306562 A1
(43) Pub. Date: Sep. 29, 2022

(54) METHOD FOR PRODUCING LEVULINIC ACID IN MOLTEN SALT HYDRATE FROM CELLULOSE HYDROLYSIS

(71) Applicant: Shandong University of Technology, Zibo (CN)

(72) Inventors: Hongyou CUI, Zibo (CN); Jinghua WANG, Zibo (CN); Yuan ZHANG, Zibo (CN); Zhihe LI, Zipo (CN); Weiming YI, Zibo (CN); Ming WANG, Zibo (SD)

(73) Assignee: Shandong University of Technology, Zibo (CN)

(21) Appl. No.: 17/611,811

(22) PCT Filed: Dec. 8, 2020
(86) PCT No.: PCT/CN2020/134566

§ 371 Date: Nov. 16, 2021

(30) Foreign Application Priority Data
Dec. 12, 2019 (CN) 201911292505.9

(51) Int. Cl. C07C 51/00; C07C 51/48

(52) U.S. Cl. CPC C07C 51/00; C07C 51/48

(57) ABSTRACT

The disclosure relates to a method for producing levulinic acid in a molten salt hydrate from cellulose hydrolysis. An inorganic molten salt hydrate was prepared by mixing an inorganic salt with water, cellulose is added and stirred to dissolve, a solid catalyst is added and heated up for reaction to obtain a reactant, the reactant is cooled and subjected to a separation to obtain the levulinic acid, and the inorganic molten salt hydrate and the solid catalyst obtained after the separation are recycled, wherein the inorganic salt is one or more selected from the group consisting of LiCl, LiBr, CaBr₂, Ca(NO₃)₂, LiNO₃ and KNO₃.

### CROSS REFERENCE TO RELATED APPLICATION(S)

This patent application claims the benefit and priority of Chinese Patent Application No. 201911292505.9, filed on Dec. 12, 2019, the disclosure of which is incorporated by reference herein in its entirety as part of the present application.

### TECHNICAL FIELD

The present disclosure belongs to the technical field of biomass resource utilization, and specifically relates to a method for producing levulinic acid in a molten salt hydrate from cellulose hydrolysis.

### BACKGROUND ART

Fossil energy is increasingly depleted, and it is urgent to explore alternative resources. Biomass is one of the renewable alternatives to fossil energy on the earth, which is mainly composed of lignin, cellulose and hemicellulose, and the cellulose accounts for about 40-50%. Converting cellulose into chemicals is one of the main ways of biomass utilization. Levulinic acid is an important platform compound derived from biomass conversion, which has extensive applications in the fields of food, medicines, pesticides, chemicals and so on. In recent years, researchers around the world have done a great deal of effort in the preparation of levulinic acid from cellulose.

Zuo et al. explored sulfonated chloromethyl polystyrene solid acid to catalyze the conversion of cellulose to levulinic acid (LA), in which 90% (w/w) γ-valerolactone and 10% (w/w) water was used as a mixed solvent, obtaining the best LA yield of 65.5% after reacting 10 h.

Yang et al. reported a selective conversion of microcrystalline cellulose, where 5% (w/w) sodium chloride solution was added under hydrothermal conditions, a resin-treated iron solid catalyst was used as catalyst, 90.9% of the microcrystalline cellulose was converted to LA at 200° C. for 5 h, and the yield of LA was 33.3%.

Wang Pan et al. investigated the conversion of cellulose to LA using a solid acid SO42-/TiO2 and ferric chloride as the catalyst, and discussed the effects of reaction temperature, reaction time, catalyst dosage and solid-liquid ratio on the yield. Experimental results showed that LA yield is 25.52% at relatively optimal process conditions at 220° C. for 15 min, with a catalyst dosage of m (cellulose): m (catalyst)=2:1 and a solid-liquid ratio of 1:15.

Han et al. used γ-valerolactone as a solvent and a lignin-based solid catalyst to catalyze cellulose for preparation of levulinic acid, where the reaction was conducted at 180° C. for 120 min, and the yield of levulinic acid was 35.64%.

Khan et al. used indium chloride dinuclear ionic liquid as reaction system and catalyst to catalyze the hydrolysis of cellulose to levulinic acid in one step. The reaction is conducted under strong acid conditions at 100° C. for 3.0 h, and the yield of levulinic acid was of 55%.

Chinese patent CN107268313A disclosed a method for hydrolysis of lignocellulose with carbon-based solid acid catalyst under microwave-promotion. In this patent, lignocellulose was soaked in 40% ZnCl2 solution, firstly. Coupling of ZnCl2 and microwave radiation promoted the hydrolysis of lignin and cellulose, and at same time promoted the breaking of hydrogen bonds of the cellulose, thereby improving the degradation of cellulose. Degradation yield of cellulose was not mentioned. In this method, 40% ZnCl2 solution cannot dissolve cellulose, and hydrolysis of cellulose is still a reaction of solid-solid which is solid cellulose using solid acid catalyst, resulting in low the reaction rate.

Chinese patent CN103435577A disclosed a method for preparing levulinic acid or co-producing γ-valerolactone from biomass. In the method, γ-valerolactone aqueous is used as solvent to dissolve cellulose and hemicellulose, and a solid acid is used as catalyst to prepare levulinic acid or further prepare γ-valerolactone by adding hydrogenation catalyst. Although this method solves the problem of cellulose dissolution, the highest yield of levulinic acid was of only 60%.

Chinese patent CN104529752A disclosed a process for preparing levulinic acid by continuous hydrolysis of cellulose in ionic liquid-water medium. In the process one ionic liquid was used as solvent and another ionic liquid as catalyst. Albeit the process realizes dissolution and homogeneous reaction of cellulose, the separation of ionic liquid as well as the high cost is still challenging. The reaction was conducted at preheating temperature of 290-310° C., reaction temperature of 200-220° C. and reaction pressure of 4-5 MPa, and the yield of levulinic acid was 72.1%.

Cellulose can not dissolve in water and conventional organic solvents, which results in low hydrolysis rate, high reaction temperature, long reaction time, and low yield of the target product. mineral acid catalysts can significantly accelerate the conversion of cellulose; however, it is difficult for separation, recycling and subsequent treatments of mineral acid, resulting in waste of resources and environmental hazards.

### SUMMARY

The present disclosure is intended to provide a method for producing levulinic acid from cellulose in a molten salt hydrate. The method of present disclosure could greatly improve the conversion rate and yield of levulinic acid from cellulose hydrolysis, products obtained by the same are easy to be separated, and reaction solvents and catalysts could be recycled.

A method for producing levulinic acid in a molten salt hydrate from cellulose hydrolysis includes preparing an inorganic salt hydrate by dissolving an inorganic salt in water, adding cellulose and stirring to dissolve, adding a solid catalyst and heating for reaction; after the reaction is completed, cooling the resulting reactant and subjecting the cooled reactant to a separation to obtain the levulinic acid, and recycling the inorganic molten salt hydrate and the solid catalyst obtained after the separation, wherein the inorganic salt is one or more selected from the group consisting of LiCl, LiBr, CaBr₂, Ca(NO₃)₂, LiNO₃ and KNO₃.

In the present disclosure, a molar ratio of the water to the inorganic salt may be (1-6):1.

In the present disclosure, 10-50 g of the cellulose may be added to every 1 L of the inorganic molten salt hydrate.

In the present disclosure, the solid catalyst may be one or two selected from the group consisting of Nb—Ce/SBA-15 and Nb—CeP/SBA-15.

In the present disclosure, a mass ratio of the solid catalyst to the cellulose may be 1:(1-5).

In the present disclosure, optionally the heating for reaction may be conducted after adding the solid catalyst and an extractant.

In the present disclosure, the extractant may be one or more selected from the group consisting of methyl isobutyl ketone (MIBK), n-butanol, ethyl acetate and octanol.

In the present disclosure, a volume ratio of the extractant to the inorganic molten salt hydrate may be (0-3):1.

In the present disclosure, the reaction may be conducted at 150-200° C.

In the present disclosure, the reaction may last for 30-120 min.

The present disclosure provides an efficient, fast and one-step cellulose hydrolysis method to produce levulinic acid.

In the present disclosure, a one-pot method for producing levulinic acid in a molten salt hydrate from cellulose hydrolysis is specifically conducted as follows: the inorganic molten salt hydrate is pre-prepared by dissolving an inorganic salt in a certain proportion of water, then cellulose is added, heated and stirred to dissolve; when the cellulose is completely dissolved, a catalyst and an extractant is added and then reacted for a certain time at a certain temperature, and then cooled down; the resulting reactant is subjected to a solid-liquid and organic phase separation to obtain an organic phase containing levulinic acid, wherein the inorganic molten salt hydrate and the solid catalyst are recycled.

In the present disclosure, the reaction can be conducted under normal pressure or low pressure.

The present disclosure has the following beneficial effects.

(1) In the present disclosure, on one hand, the inorganic molten salt hydrate is used as solvent to dissolve cellulose; on the other hand, the inorganic molten salt hydrate has a temperature-rising effect, which enables the reaction to be conducted under mild conditions such as normal pressure or low pressure; at the same time, both cations and anions in the inorganic molten salt hydrate could catalyze the hydrolysis of the cellulose and intermediate products.

(2) In the present disclosure, a heterogeneous catalyst is used, which is easy to separate and reusable; both carrier and active components of the heterogeneous catalyst have catalytic effects.

(3) In the present disclosure, the hydrolysis of cellulose is a coupled catalytic reaction of homogeneous catalyst and heterogeneous catalyst. The synergistic catalysis of anions and cations in the inorganic molten salt hydrate with the solid catalyst could greatly improve reaction rate, selectivity and yield.

(4) In the present disclosure, the extractant could extract an intermediate product into the organic phase in time, reducing the occurrence of side reactions.

(5) In the present disclosure, the inorganic molten salt hydrate and the solid catalyst could be separated by settlement after the reaction, and could be recycled.

(6) In the present disclosure, yield of levulinic acid produced from cellulose one-step hydrolysis is not less than 90%.

### DETAILED DESCRIPTION OF THE EMBODIMENTS

Hereinafter, the present disclosure will be further described in conjunction with examples.

### Example 1

(1) First a certain amount of LiBr was weighed, and then water was added thereto based on the 3:1 molar ratio of water:LiBr while stirring until LiBr was dissolved, obtaining LiBr.3H₂O.

(2) 0.1 g of cellulose was added to 5 mL of LiBr.3H₂O, stirred to dissolve, and 0.1 g of Nb—Ce/SBA-15 and 5 mL of MIBK were added.

(3) The resulting mixture was heated up to 150° C., and subjected to a reaction at this temperature for 120 min.

(4) After reaction, the resulting reactant was cooled, and centrifuged, and then detected. Separated LiBr.3H₂O and Nb—Ce/SBA-15 were recycled.

After detection, the yield of levulinic acid is 91.3%.

### Example 2

(1) First a certain amount of LiCl was weighed, and then water was added thereto based on the 2:1 molar ratio of water:LiCl while stirring until LiCi was dissolved, obtaining LiCl.2H₂O.

(2) 0.2 g of cellulose was added to 5 mL of LiCl.2H₂O, stirred to dissolve, and 0.1 g of Nb—Ce/SBA-15 and 10 mL of MIBK were added.

(3) The resulting mixture was heated up to 175° C., and subjected to a reaction at this temperature for 90 min.

(4) After reaction, the resulting reactant was cooled, and centrifuged, and then detected. Separated LiCl.2H₂O and Nb—Ce/SBA-15 were recycled.

After detection, the yield of levulinic acid is 92.7%.

### Example 3

(1) First a certain amount of LiCl was weighted, and then water was added thereto based on the 3:1 molar ratio of water:salt while stirring until LiCl was dissolved, obtaining LiCl.3H₂O.

(2) 0.1 g of cellulose was added to 5 mL of LiCl.3H₂O, stirred to dissolve, and 0.1 g Nb—Ce/SBA-15 and 5 mL of MIBK were added.

(3) The resulting mixture was heated up to 160° C., and subjected to a reaction at this temperature for 120 min.

(4) After reaction, the resulting reactant was cooled, and centrifuged, and then detected. Separated LiCl.3H₂O and Nb—Ce/SBA-15 were recycled.

After detection, the yield of levulinic acid is 91.2%.

### Example 4

(1) First a certain amount of LiBr was weighed, and then water was added thereto based on the 4:1 molar ratio of water:LiBr while stirring until LiBr was dissolved, obtaining LiBr.4H₂O.

(2) 0.1 g of cellulose was added to 5 mL of LiBr.4H₂O, stirred to dissolve, and 0.1 g of Nb—CeP/SBA-15 and 5 mL of octanol were added.

(3) The resulting mixture was heated up to 175° C., and subjected to a reaction at this temperature for 60 min.

(4) After reaction, the resulting reactant was cooled, and centrifuged, and then detected. Separated LiBr.4H₂O and Nb—CeP/SBA-15 was recycled.

After detection, the yield of levulinic acid is 93.1%.

### Example 5

(1) First a certain amount of LiBr was weighed, and then water was added thereto based on the 2:1 molar ratio of water:LiBr while stirring until LiBr was dissolved, obtaining LiBr.2H₂O. A certain amount of Ca(NO₃)₂ was weighed, and water was added thereto based on the 2:1 molar ratio of water:Ca(NO₃)₂ while stirring until Ca(NO₃)₂ was dissolved, obtaining Ca(NO₃)₂.2H₂O.

(2) 0.1 g of cellulose was added to 5 mL of LiBr.2H₂O+Ca(NO₃)₂.2H₂O (with a volume ratio of LiBr.2H₂O to Ca(NO₃)₂.2H₂O of 1:1), stirred to dissolve, and 0.1 g of Nb—CeP/SBA-15 and 5 mL of MIBK were added.

(3) The resulting mixture was heated up to 160° C., and subjected to a reaction at this temperature for 50 min.

(4) After reaction, the resulting reactant was cooled, and centrifuged, and then detected. Separated LiBr.2H₂O+Ca(NO₃)₂.2H₂O and Nb—CeP/SBA-15 were recycled.

After detection, the yield of levulinic acid is 92.2%.

### Example 6

(1) First a certain amount of LiCl was weighed, and then water was added thereto based on the 2:1 molar ratio of water:LiCl while stirring until LiCl was dissolved, obtaining LiCl.2H₂O. A certain amount of LiNO₃ was weighed, and then water was added thereto based on the 2:1 molar ratio of water:LiNO₃ while stirring until LiNO₃ was dissolved, obtaining LiNO₃.2H₂O.

(2) 0.2 g of cellulose was added to 5 mL of LiCl.2H₂O+LiNO₃.2H₂O (with a volume ratio of LiCl.2H₂O to LiNO₃.2H₂O of 1:1), stirred to dissolve, and 0.1 g of Nb—CeP/SBA-15 was added.

(3) The resulting mixture was heated up to 175° C., and subjected to a reaction at this temperature for 30 min.

(4) After reaction, the resulting reactant was cooled, and centrifuged, and then detected. Separated LiCl.2H₂O+LiNO₃.2H₂O and Nb—CeP/SBA-15 were recycled.

After detection, the yield of levulinic acid is 91.3%.

### Example 7

(1) First a certain amount of LiCl was weighed, and then water was added thereto based on the 2:1 molar ratio of water:LiCl while stirring until LiCl was dissolved, obtaining LiCl.2H₂O. A certain amount of KNO₃ was weighed, and then water was added thereto based on the 1:1 molar ratio of water:KNO₃ while stirring until KNO₃ was dissolved, obtaining KNO₃.H₂O.

(2) 0.2 g of cellulose was added to 5 mL of LiCl.2H₂O+KNO₃.H₂O (with a volume ratio of LiCl.2H₂O to KNO₃.H₂O of 1:1), stirred to dissolve, and 0.1 g of Nb—CeP/SBA-15 and 10 mL of MIBK were added.

(3) The resulting mixture was heated up to 175° C., and subjected to a reaction at this temperature for 30 min.

(4) After reaction, the resulting reactant was cooled, and centrifuged, and then detected. Separated LiCl.2H₂O+KNO₃.H₂O and Nb—CeP/SBA-15 were recycled.

After detection, the yield of levulinic acid is 93.6%.

### Example 8

(1) First a certain amount of CaBr₂ was weighed, and then water was added thereto based on the 2:1 molar ratio of water:CaBr₂ while stirring until CaBr₂ was dissolved, obtaining CaBr₂2H₂O.

(2) 0.2 g of cellulose was added to 5 mL of CaBr₂ 2H₂O, stirred to dissolve, and 0.1 g of Nb—CeP/SBA-15 was added.

(3) The resulting mixture was heated up to 175° C., and subjected to a reaction at this temperature for 30 min.

(4) After reaction, the resulting reactant was cooled, and centrifuged, and then detected. Separated CaBr₂ 2H₂O and Nb—CeP/SBA-15 were recycled.

After detection, the yield of levulinic acid is 90.4%.

### Comparative Example 1

(1) 0.1 g of cellulose was added to 5 mL of H₂O, stirred to dissolve, and 0.1 g Nb—CeP/SBA-15 was added.

(2) The resulting mixture was heated up to 175° C., and subjected to a reaction at this temperature for 30 min.

(3) After reaction, the resulting reactant was cooled, and centrifuged, and then detected.

After detection, the yield of levulinic acid is 33.8%.

### Comparative Example 2

(1) 0.1 g of cellulose was added to 5 mL of H₂O, stirred to dissolve, and 0.1 g of Nb—Ce/SBA-15 and 10 mL of MIBK were added.

(2) The resulting mixture was heated up to 175° C., and subjected to a reaction at this temperature for 30 min.

(3) After reaction, the resulting reactant was cooled, and centrifuged, and then detected.

After detection, the yield of levulinic acid is 30.2%.

Detection results of Examples 1-8 and Comparative Examples 1-2 are shown in Table 1.

TABLE 1
Results of Examples 1-8 and Comparative Examples 1-2

| | | | | Reaction | | Levulinic |
|---|---|---|---|---|---|---|
| | Inorganic molten salt | | | temperature | Retention | acid |
| | hydrate composition | Catalyst | Extractant | (° C.) | time (min) | yield, % |
| Example 1 | LiBr•3H₂O | Nb-Ce/SBA-15 | MIBK | 150 | 120 | 91.3 |
| Example 2 | LiCl•2H₂O | Nb-Ce/SBA-15 | MIBK | 175 | 90 | 92.7 |
| Example 3 | LiCl•3H₂O | Nb-Ce/SBA-15 | MIBK | 160 | 120 | 91.2 |
| Example 4 | LiBr•2H₂O | Nb-CeP/SBA-15 | Octanol | 175 | 60 | 93.1 |
| Example 5 | LiBr•2H₂O + Ca(NO₃)₂•2H₂O | Nb-CeP/SBA-15 | MIBK | 160 | 50 | 92.2 |
| Example 6 | LiCl•2H₂O + LiNO₃•2H₂O | Nb-CeP/SBA-15 | None | 175 | 30 | 91.3 |
| Example 7 | LiCl•2H₂O + KNO₃•H₂O | Nb-CeP/SBA-15 | MIBK | 175 | 30 | 93.6 |
| Example 8 | CaBr₂•2H₂O | Nb-CeP/SBA-15 | None | 175 | 30 | 90.4 |
| Comparative | H₂O | Nb-CeP/SBA-15 | None | 175 | 30 | 33.8 |
| Example 1 | | | | | | |
| Comparative | H₂O | Nb-Ce/SBA-15 | MIBK | 175 | 30 | 30.2 |
| Example 2 | | | | | | |

It can be seen from Table 1 that in the present disclosure, under a combined action of the inorganic molten salt hydrate, the extractant and the solid catalyst, yields of levulinic acid are all not less than 90%.

What is claimed is:

1. A method for producing levulinic acid in a molten salt hydrate from cellulose hydrolysis, comprising preparing an inorganic molten salt hydrate by mixing water with an inorganic salt, adding cellulose and stirring to dissolve, adding a solid catalyst and heating up for reaction to obtain a reactant, cooling, and subjecting the cooled reactant to a separation to obtain the levulinic acid, and recycling the inorganic molten salt hydrate and the solid catalyst obtained after the separation, wherein the inorganic salt is one or more selected from the group consisting of LiCl, LiBr, CaBr₂, Ca(NO₃)₂, LiNO₃ and KNO₃.

2. The method for producing levulinic acid in a molten salt hydrate from cellulose hydrolysis according to claim 1, wherein a molar ratio of the water to the inorganic salt is (1-6):1.

3. The method for preparing levulinic acid in a molten salt hydrate from cellulose hydrolysis according to claim 1, wherein 10-50 g of the cellulose is added to every 1 L of the inorganic molten salt hydrate.

4. The method for producing levulinic acid in a molten salt hydrate from cellulose hydrolysis according to claim 1, wherein the solid catalyst is one or two selected from the group consisting of Nb—Ce/SBA-15 and Nb—CeP/SBA-15.

5. The method for producing levulinic acid in a molten salt hydrate from cellulose hydrolysis according to claim 1, wherein a mass ratio of the solid catalyst to the cellulose is 1:(1-5).

6. The method for producing levulinic acid in a molten salt hydrate from cellulose hydrolysis according to claim 1, wherein heating up for reaction is conducted after adding the solid catalyst and an extractant.

7. The method for producing levulinic acid in a molten salt hydrate from cellulose hydrolysis according to claim 6, wherein the extractant is one or more selected from the group consisting of methyl isobutyl ketone (MIBK), n-butanol, ethyl acetate and octanol.

8. The method for producing levulinic acid in a molten salt hydrate from cellulose hydrolysis according to claim 7, wherein a volume ratio of the extractant to the inorganic molten salt hydrate is (0-3):1.

9. The method for producing levulinic acid in a molten salt hydrate from cellulose hydrolysis according to claim 1, wherein the reaction is conducted at 150-200° C.

10. The method for producing levulinic acid in a molten salt hydrate from cellulose hydrolysis according to claim 1, wherein the reaction is conducted for 30-120 min.
